(19) United States
(12) Patent Application Publication
(10) Pub. No.: US 2014/0030762 A1
(43) Pub. Date: Jan. 30, 2014

(54) BIOREACTOR FOR CELL CULTURE ON A THREE-DIMENSIONAL SUBSTRATE

(71) Applicants: Valerie Deplano, Allauch (FR); Yannick Knapp, Marseille (FR); Eric Bertrand, Marseille (FR)

(72) Inventors: Valerie Deplano, Allauch (FR); Yannick Knapp, Marseille (FR); Eric Bertrand, Marseille (FR)

(73) Assignee: CENTRE NATIONAL DE LA RECHERCHE SCIENTIFIQUE- CNRS, Paris 16 (FR)

(21) Appl. No.: 13/983,176

(22) PCT Filed: Feb. 6, 2012
(86) PCT No.: PCT/EP2012/051983

§ 371 Date: Oct. 8, 2013

(30) Foreign Application Priority Data
Feb. 4, 2011 (FR) 1150906

(51) Int. Cl. C12M 1/34

(52) U.S. Cl. CPC C12M 41/44
USPC 435/70.3; 435/289.1; 435/397

(57) ABSTRACT

The invention relates to a bioreactor (1) for cell culture on a three-dimensional substrate, comprising
a culture chamber (2), the inner walls of which form a vertical duct, preferably, tapered, with a diameter that widens regularly form the duct inlet to the duct outlet, means (3, 4) enabling the culture medium to flow in said vertical duct.

The invention also relates to the advantageous use of these bioreactors in tissue engineering, for the production of tissue grafts, notably a bone or cartilage graft.

### FIELD OF THE INVENTION

The invention relates to the field of bioreactors for cell culture. In particular, the invention relates to a bioreactor (1) for cell culture on a three-dimensional substrate, comprising: a culture chamber (2), the inner walls of which form a vertical duct, preferably tapered, with a diameter that widens regularly from the duct inlet to the duct outlet, means (3, 4) enabling the culture medium to flow in said vertical duct.

The invention also relates to the advantageous use of these bioreactors in tissue engineering, for the production of tissue grafts, notably hone or cartilage grafts.

### CONTEXT OF THE INVENTION

The aim of tissue engineering is to apply the principles of biology and of engineering in order to develop functional substitutes for injured tissues. Technological developments in tissue engineering should make it possible to obtain, from patient cells, tissues cultivated in vitro that can be tolerated by the organism and replace the injured or failing tissue. The regeneration prospects offered by tissue engineering embrace many tissue types including, non-exhaustively, cardiac tissue, certain tissues of the eye (cornea), hepatic and pancreatic tissues, blood vessels and musculoskeletal tissues: muscle, bone and cartilage tissues, but also tendon and ligament tissues.

To obtain a tissue graft or organoid, it is usually necessary to seed the appropriate cells, for example progenitor cells of the targeted tissues, in porous biomaterials allowing for the development of a three-dimensional structure. This is then referred to as cell culture on a three-dimensional substrate, or three-dimensional cell culture.

For culture on a three-dimensional substrate, a so-called static culture mode can be used: this culture mode consists in dipping the cell-seeded substrate in a nutrient liquid and in placing it in a temperature-regulating incubator and gaseous mixture. The main drawback with this technology lies in the fact that the diffusive exchanges, only present here, are generally insufficient to ensure the nutrition of the cells and in particular at the heart of the substrate.

In fact, a good perfusion of the nutrient liquid through the three-dimensional substrates has been identified in the prior art as being a determining criterion for an adequate growth and development of the tissue graft in a three-dimensional culture. Also, since the traditional cultures on Petri dish or in static culture are not suited to cell cultures on a three-dimensional substrate, so-called “dynamic” culture processes, that is to say processes that involve a movement of the culture medium in the substrate, have been developed.

There are various types of “dynamic” cell culture bioreactors known in the prior art, and the most widely used are:
agitation flasks (spinner flask, wave bioreactors). In such a system, the seeded substrates are “hung” in the agitated nutrient liquid. The flask can have various forms in order to enhance the exchanges of nutrients and of products of the metabolism. The main drawback with this technology lies in the fact that the substrates have to be held on a specific support that will have to be removed before implantation. Furthermore, this culture method often results in the generation of turbulent flows in the flasks and of significant shear levels on the surface of the substrates, both of which are prejudicial to the good development of the grafts.
rotating wall bioreactors (Rotating Wall Vessels). Derived from developments of bioreactors included in space flights, these bioreactors can simulate microgravity conditions and make it possible to study the development of tissues in space. In these devices, the cell-seeded substrates are maintained in equilibrium in the air gap of two contrarotating cylinders containing the nutrient liquid. The convection movements ensured by the rotation of these walls allow for the renewal of the nutrient liquid. The main drawbacks with this technology lie in the fact that:
a) premature cell death is observed because of the impacts between the substrates and between the substrates and the wall (centrifugation),
b) there are engineering constraints (accurate control of the rotational movements requiring servo control, seals, etc.) and ergonomic constraints (assembly, dismantling, sampling, sterilization), often prohibitive for routine use because of the consequential excessive number of setting and operation parameters,
Perfusion bioreactors. In these systems, the cell-seeded porous substrates are subjected to a perfusion flow of nutrient liquid. The substrate has to be held in place in the bioreactor so that the flow can be forced therein. It is therefore imperative for the substrate to have a minimum of mechanical resistance to withstand the contrary perfusion and holding forces. These substrates are primarily of use in the case of bone tissue engineering; in this case, the culture substrate has mechanical performance characteristics very similar to the bone in culture. The main drawback with this technology lies in the fact that some substrates do not necessarily have sufficient mechanical performance characteristics to withstand significant transmural pressures (given the size of the substrates provided).

Furthermore, the American patent U.S. Pat. No. 5,320,963 describes a conical bioreactor for suspension cell culture. The device relates to the isolated or mass cell cultures cultivated without substrate, the conical part being put n place to offer a larger deposition surface for the harvesting of the cells.

Moreover, Singh et al (2007, Biotechnology and Bioengineering, Vol. 97, No 5, pp 1291-1299) have also proposed a conical bioreactor which would make it possible, according to the authors, to maintain a biomaterial in suspension, independently of its weight. However, the latter aspect is not demonstrated since the aim of the work presented in this paper is to determine the flow around a single biomaterial maintained by a rod and to demonstrate the existence of a flow inside said three-dimensional biomaterial woven with different types of weaves. Rather, the biomaterial has a large dimension compared to the duct opening and is maintained artificially in a fixed position in the conical chamber. Thus, it should be noted:
that no demonstration of cell culture is made in this study,
that the bioreactor described has only a single substrate in the culture chamber,
and that this substrate is not suspended in the culture chamber.

Porous hydrogels are materials that have very great potential when it comes to cell culture in three dimensions and they are used as replacement for many materials usually used in cell culture (coral, hydroxihapatite, titanium, etc.) because of their similarity with the native tissues (“softer” materials) and their very great biocompatibility (resorbable polysaccharide). However, these materials have mechanical characteristics that are inadequate for placement in the known bioreactors of the state of the art, and in particular the perfusion bioreactors described in the literature (held between “clamps”+application of a transmural pressure).

### OBJECTIVES OF THE INVENTION

The objective of the invention mitigate the defiencies of the prior art devices. In particular, the bioreactors according to the invention make it possible to maintain in suspension cells cultivated on a three-dimensional substrate by virtue of the balance between different hydrodynamic forces (Stokes drag, gravity and buoyancy). In practice, the inventors have shown merit in developing a bioreactor capable of generating a particular flow in the culture chamber; typically by controlling the horizontal velocity gradient, making it possible to obtain the maintaining in suspension of the biomaterials or grafts in culture.

In particular, the generation of a uniform and symmetrical flow in principle entails bringing the nutrient fluid into ducts of large dimensions in order to get away from any singularity in the flow, equally due to the walls and the geometrical singularities such as bends, section variations or even the flow generation systems. Such dimensions are not then realistic in the case of cell culture devices because of the high cost of the nutrient fluids. Another objective of the invention is therefore to develop a device using a smaller volume of fluids while allowing for an adequate flow in order to achieve the first objective.

Moreover, tests, transcribed in the literature on other bioreactors using sustentation of the substrates (rotating wall vessel bioreactors), have shown that the cell culture was potentially degraded by the interactions (impacts) between the substrates and the walls of the bioreactor. Thus, in order to limit such interactions, another objective of the invention is to develop a device generating a flow that can keep the substrates in culture far from the walls, suitable in particular for cell culture on substrates of porous hydrogel type.

Yet another objective of the invention is to allow for a perfusion of the substrates so as to obtain an optimum and uniform growth of the grafts in the bioreactor.

An additional objective of the invention is to provide a bioreactor for cell culture on a substrate in suspension that requires the minimum of human intervention during a culture time of the order of a few days to several weeks.

### BRIEF DESCRIPTION OF THE INVENTION

The inventors have shown merit in having developed a bioreactor that satisfies all the objectives identified above. In particular, such a bioreactor allows for the culture of tissue grafts held in suspension and perfused by the culture medium. It is particularly suited to the culture of bone or cartilage grafts on soft porous substrates, notably on porous hydrogels.

In order to put in place this technology, the inventors have produced a bioreactor comprising an appropriate flow device for the culture medium in the culture chamber.

They have also shrewdly chosen a form for the culture chamber which, combined with the flow mode, makes it possible to prevent the materials in suspension (grafts) impacting on the walls of the culture chamber.

Thus, the invention, as defined in the claims, relates firstly to a bioreactor for cell culture on a three-dimensional substrate, wherein it comprises

a) a culture chamber, the inner walls of which form a vertical duct, preferably tapered, with a diameter that widens regularly from the duct inlet (for example the bottom inlet) to the duct outlet (for example the top outlet),

b) means enabling the culture medium to flow (for example from bottom to top) in said vertical duct.

According to a preferred embodiment, the bioreactor also comprises pumping means allowing for a pulsed flow of the culture medium.

According to a second preferred embodiment, the bioreactor comprises means allowing for an annular flow of the culture medium in the culture chamber.

### BRIEF DESCRIPTION OF THE DRAWINGS

FIG. 1 shows a schematic overview of the various parts of a bioreactor (1).

FIG. 2 shows a detailed view of a culture chamber (2).

FIG. 3 shows an exploded detail view of an upstream flow-creating device (3).

FIG. 4 shows a partial cross-sectional view of a bioreactor according to the invention comprising the culture chamber (2), the upstream flow device (3) and the downstream flow device (6).

FIG. 5 shows a detailed view of an upstream flow-creating device (3) and shows the path of the fluids through the device.

FIG. 6 shows a partial cross-sectional view of a bioreactor according to the invention and shows the path of the fluids through the device.

FIG. 7 shows the cell viability revealed by live/dead coloring of the ADSCs inside the matrix, after 5 days of dynamic culture A) on the edges of the porous matrix or B) at the center (×10 zoom). Scale bar: 200 μm, and the relative expression of the rates of mRNA markers specific to the ALP (C), OPN (D), OCN (E) and Cx43 (F) bone.

### DETAILED DESCRIPTION OF THE INVENTION

The invention will now be described with reference to the figures mentioned above, essentially in the case of an appropriate bioreactor for the culture of mammal cells on a porous three-dimensional substrate, such as porous hydrogels. Obviously, other types of cells and of three-dimensional substrates can be used in the bioreactor according to the invention.

The term “bioreactor” is used to denote a device that makes it possible to grow biological cells in a preferably sterile medium.

For example, the biological cells that can be cultivated in bioreactors are prokaryotic or eukaryotic cells, and notably microorganisms, unicellular eukaryotic or prokaryotic organisms, such as bacteria, archaea, yeasts or mushrooms, or cells of pluricellular organisms and notably mammal cells, notably embryonic or somatic cells, or stem cells, for example mesenchymatous stem cells of mammals or their derivatives.

The terms “three-dimensional substrate” and “biomaterial” (the two terms being used without differentiation), should be understood to mean an artificial or natural material, that allows for the three-dimensional growth of the cells, and notably the growth of organoids from stem cells comprising the differentiation of the stem cells into different cell types within the biomaterial. These biomaterials have to have a porous structure, favoring the growth of the cells while allowing for a good perfusion of the nutrient liquids within the structure. For tissue engineering, it is generally considered that an average pore size of between 200 and 400 μm is optimum for favoring the penetration of the cells into the implant but also the formation after implantation of a vascular network.

The biomaterials include, without being limiting, the porous biomaterials of polymeric or ceramic nature, metallic structures of titanium, tantalum or nitinol type. The porous biomaterials of polymeric type include, for example, polylactic acid (PLA), polyglycolic acid (PGA), polylactic co-glycolic acid (PLAGA), hyaluronic acid or even polycaprolactone (PCL). The synthetic ceramics (hydroxyapatite and tricalcic phosphates) or natural ceramics (coral and mother-of-pearl) can also be used as biomaterial, notably for the culture of bone organoids.

Other, so-called “resorbable” biomaterials have been developed and comprise materials of biological origin (for example, alginate, collagen or even fibrin gels).

A preferred biomaterial mode that can be used in the bioreactors according to the invention are “hydrogels” or “porous hydrogels”. These porous hydrogels are, for example, based on polymers chosen from among polyethylene glycol (PEG), polyvinyl alcohol) (PVA) and poly(2-hydroxy ethyl methacrylate) (pHEMA). These materials may also include different additives, for example different collagens, chitosan, etc., promoting the specific phases in the cell development or modifying the physical-chemical properties of the material (see in particular the publication “Tissue Engineering: Fundamentals and applications”, 2006 by Yoshito Ikada in the collection Interface Science and Technology Ed. Academic Press. Chapter 3 Section 4 Surface Modification of Biomaterials and Cell interactions).

In a preferred embodiment, the hydrogels used are chosen from among polysaccharide-based hydrogels, and notably those based on Pullulan as described in European Cells and Materials Vol. 13. Suppl. 1, 2007 (page 50).

The terms “organoids” or “graft” should be understood to mean a three-dimensional structure consisting of at least one biomaterial and biological cells capable of proliferating on the biomaterial, for example to form a graft which can he transplanted onto a patient. The grafts or organoids cultivated in the bioreactor according to the invention can advantageously be of small dimensions, for example, having a maximum section of between 2 and 20 mm, for example between 4 and 15 mm, even between 2 and 10 mm.

The bioreactor according to the invention is characterized in that it comprises:

a) a culture chamber (2), the inner walls of which form a vertical duct, preferably tapered, with a diameter that widens regularly from the duct inlet (for example the bottom inlet) to the duct outlet (for example the top outlet),

means (3, 4) enabling the culture medium to flow (for example from bottom to top) in said vertical duct.

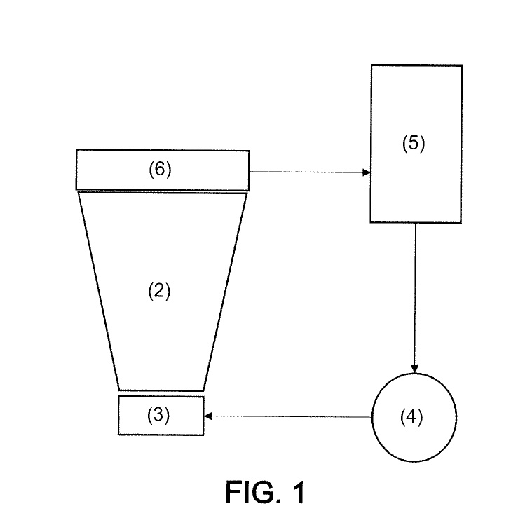

Typically, a bioreactor (1) according to the invention may comprise in particular the five elements as represented in FIG. 1:
a culture chamber (2),
art upstream flow-creating device (3),
pumping means (4),
a culture medium tank (5),
a downstream flow-creating device (6),
   the different parts being interconnected by ducts. It is also possible to envisage an optional metrology (pressure/flow rate/temperature).

Naturally, variants are possible, the bioreactor may notably comprise a plurality of culture chambers, or one culture chamber divided into a plurality of compartments, for example by gratings or semi-tight walls with a porosity that makes it possible
to allow the nutrient medium to circulate,
to retain the grafts or biomaterials in separate compartments,
and thus to separately cultivate different cell types or grafts.

The Culture Chamber (2)

It comprises a main body, the inner walls of which form a vertical duct through which the culture medium flows. The duct, whose geometrical axis is vertical, has a preferably circular cross section, with a diameter that widens regularly from the duct inlet to the duct outlet.

The duct inlet and outlet are determined by the direction of flow of the culture medium in the culture chamber.

The advantageous form of the vertical duct of the culture chamber of the bioreactors according to the invention helps in the self-regulation of the sustentation of the biomaterials or grafts by making it possible to establish a balance between the forces of drag (flow of the fluid around the hydrogels), of gravity (weight of the substrate) and the resultant buoyancy (buoyancy linked to the difference in density between the solid and the fluid). This balance has been described for example in industrial applications of flow metrology (“rotameter” type flow meter), but in such applications, the single object in sustentation has dimensions very close to those of the duct.

In a specific and preferred embodiment, appropriate to the use of substrates in culture with a density greater than that of the culture medium, the duct has a cross section with a diameter that widens regularly from the bottom duct inlet to the top duct outlet.

In normal culture conditions in such an embodiment, the cultivated biomaterials (biomaterials, grafts, etc.) are driven vertically by the high velocities present in the small section of the cone until they arrive in a zone of lower velocities (in the larger section) where the drag forces are proportionally lower (gravity once again becomes the predominant force) which causes the cultivated biomaterials to drop toward the zone of higher velocity where the lifts to the zone of lower velocity recommence. There thus follows a phenomenon of alternate vertical circulation in three dimensions in the body of the culture chamber. This movement promotes the renewal of the culture medium on the surface of the cultivated biomaterials and, consequently, permanently ensures a concentration gradient between the interior and the surface of the support that is as favorable as possible (diffusive effects maximized). Moreover, the porosity of the cultivated biomaterial allows for the passage of a fluid. The convection of the fluid in the biomaterial is then ensured by the existence of a pressure gradient between the bottom and the top faces of the substrate. This convection is reinforced on the one hand by the alternate circulation movements described above and, on the other hand, by the application of a pulsed flow in the culture chamber. The cultivated biomaterial is then advantageously perfused (in proportion to its permeability and to the pressure gradient between the bottom and top walls of each biomaterial).

Alternatively, it is also possible to envisage a device comprising a vertical duct having a vertical section with a diameter that widens regularly from the top duct inlet to the bottom duct outlet. This embodiment is more particularly appropriate for the culture of porous substrates with a density less than that of the fluid (for example, when the substrate includes air bubbles).

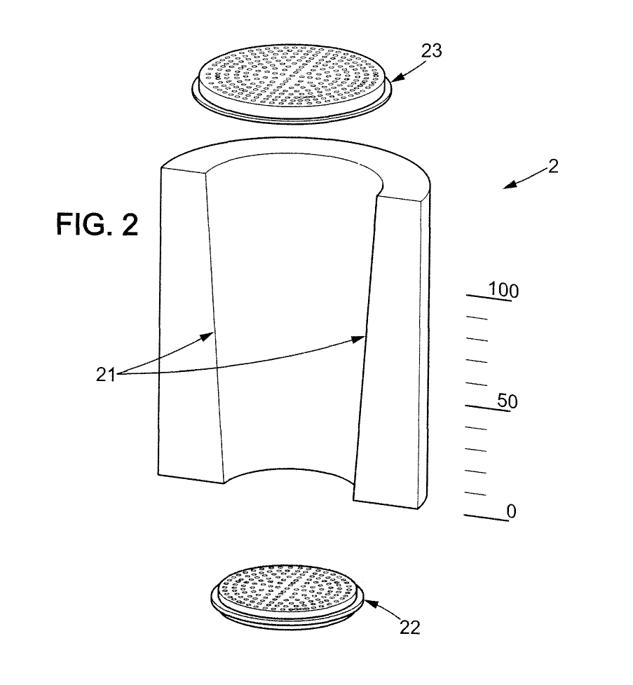

In a preferred embodiment, the culture chamber comprises a cylindrical body pierced by a tapered duct, in frustoconical form. The tapered duct is, for example, inverted as represented in FIG. 2.

In the culture methods implemented with the bioreactors of the present invention, the cultivated biomaterials are generally of small dimensions relative to the culture chamber, for example with a dimension less than 20 mm, for example between 4 and 15 mm, for an inlet diameter of the vertical duct (smaller section) which can, for example, be between 3 cm and 10 cm.

In a preferred embodiment, a culture chamber will be chosen that comprises a duct of tapered form with an angle at the apex that does not exceed 8°.

The diameter of the duct inlet cross section (smaller section) can, for example, without being limiting, he between 3 cm and 10 cm, the height of the vertical duct between 5 cm and 30 cm and the diameter of the duct outlet cross section (larger section), between 3 cm and 15 cm. Thus, the culture chamber can contain a volume of culture of approximately 35 ml to 4 liters.

A person skilled in the art will select the most appropriate material for the culture chamber, notably from those known from the prior art for the production of bioreactor culture chambers. These materials notably include glass, transparent polymers such as PE, PET, PVC, PS, PP, PMMA, PEI and ABS. It is preferably a sterilizable material.

For example, the pierced cylindrical body of a tapered duct of the culture chamber is made of a material of sterilizable PolyEtherlmide type.

In a preferred embodiment, the material is a transparent material. It thus makes it possible to visually set the conditions for the sustentation of the cultivated biomaterials. Their position in the culture chamber is thus controlled. This is an advantageous aspect of the invention because it allows non-experts to establish an adequate flow and to correct it throughout the evolution of the cell culture (in the case of the fabrication of bone grafts, the cells fabricate an extracellular matrix and a calcification which consequentially increases the gravitational force). The use of a transparent material for the culture chamber can also be an advantage in the case of the use of the bioreactor in applications other than cell culture for tissue engineering, in particular those requiring the activation of photosynthesis processes.

In a specific embodiment, the bioreactor according to the invention comprises:
a culture chamber (2), the inner walls (21) of which delimit a vertical duct with a diameter that widens regularly from the duct inlet to the duct outlet, preferably tapered,
an inlet grating (22) placed in the smaller diameter upstream part (for example the bottom part) of the vertical duct promoting an annular flow of the culture medium,
if appropriate, an outlet grating (23) placed in the larger diameter downstream part (for example the top part) of the vertical duct preventing the grafts from circulating in the rest of the device,
an upstream flow-creating device (3) upstream of the inlet grating promoting the annular flow in the vertical duct,
pumping means (4), preferably for a pulsed flow,
if appropriate, a culture medium tank (5) downstream of the culture chamber (and/or upstream of the pump),
if appropriate, a downstream flowing device (6) downstream of the outlet grating.

In a specific embodiment, the bioreactor according to the invention comprises an inlet grating (22) placed upstream of the body. It is, for example, a perforated disc which promotes the creation of a flow with a velocity profile of annular type.

The expression “annular flow” should he understood to mean a flow in which the flow rate is greater through individual surfaces situated at the periphery compared to the flow rate generated through individual surfaces situated at the center of the duct.

The type of flow thus generated, unlike in the case of parabolic velocity profiles, makes it possible to maintain the substrates at the center of the tapered part of the culture chamber. In practice, in the case of parabolic velocity profiles, the substrates have a tendency to migrate toward the walls by virtue of the velocity gradient between the center and the periphery of the flow section. The diameter and the distribution of the perforations ensures that the substrates are maintained during the placement and start-up of the bioreactor but also more generally in the event of shutdown of the pumping system.

Alternatively, the generation of an annular flow can be performed using concentric cylindrical ducts instead of the perforated disc.

In a preferred embodiment, the inlet grating is a perforated grating having orifices, preferably with a diameter less than 6 mm, for example from 2 to 5 mm, distributed in such a way that the flow is faster in the regions close to the walls than at the center.

The bioreactor according to the invention may also comprise a top grating (23) placed downstream of the pierced cylindrical body of a tapered duct, it is mainly a safety device, the function of which is to prevent the accidental passage of the substrates into the rest of the hydrodynamic circuit. For example:, it may consist of a perforated disc, but with no particular distribution of the perforations. The diameter of the perforations is, however, adapted to the size of the substrates placed in culture in order to prevent their possible circulation in the rest of the installation.

The Pumping Means (4)

The bioreactor comprises pumping means that make it possible to obtain a vertical flow, from the smallest section to the largest section of the vertical duct of the culture chamber, for example a pulsed flow from bottom to top in the vertical duct. Any type of pump conventionally used in dynamic bioreactors can be envisaged.

A pump will preferably be chosen that makes it possible to avoid heating the culture medium for the control of the optimum culture temperature.

In a particularly advantageous embodiment, a pump will be chosen that makes it possible to obtain a pulsed flow.

The expression “pulsed flow” should be understood to mean a flow that exhibits, at short and regular time intervals, an acceleration phase followed by a deceleration phase. In a preferential embodiment, the frequency of the pulsings is between 0.05 and 10 Hz depending on the size of the bioreactor, for example of the order of 1 Hz thus reproducing the frequencies observed in the cardiovascular flows of an adult.

The pulsing of the flow:
makes it possible to avoid stagnation, in a particular area of the culture chamber, of the cultivated biomaterial, for example, the tissue graft, by preventing the creation of a permanent, flow,
reinforces the perfusion of the biomaterial by the combined effects of two phenomena: the first relates to the very amplitude of the flow which cannot be kept at high values permanently; in such a case, a bioreactor of large dimensions would be needed, which is contrary to the low volume constraints already discussed above. Thus, by virtue of the inertia of the grafts in sustentation in the bioreactor, the pulsed nature of the flow makes it possible to intermittently apply high perfusion flow rate values. The second phenomenon is linked to the generation of a wake downstream of the objects in relative displacement in a fluid. The pulsed flow makes it possible to not maintain the same wake over time and consequently prevents the appearance of these areas of stasis.
makes the morphology of the grafts uniform, the pulsed flow having a tendency to regularly turn over the graft in culture in the bioreactor,
by its nature simulates what happens in living organisms.

In a specific embodiment, in which a pulsed flow is used, it may be advantageous to provide a non-return valve at the pump outlet, or any means that make it possible to avoid backward flows, notably when the culture method is started up. In practice, on start-up, the three-dimensional substrates are placed on the bottom grating 22 and are then likely to be sucked up when the pump is started up for a pulsed flow.

The Upstream Flow-Creating Device (3)

This is a device for generating conditions favorable to the annular flow produced in the culture chamber. It is placed upstream of the culture chamber.

The device according to the present invention consists of a series of geometrical singularities (section variations and changes of direction of the flow) that make it possible to convert a tangential flow at the inlet of the device to render it axial with a velocity profile with axial symmetry (X to 90%) at the inlet grating of the culture chamber.

In a specific embodiment in which an upward vertical flow of the culture medium is provided in the culture chamber, the upstream flow-creating device (3) comprises:

a) a first flow zone (31), in the form of a ring, the axis of which is placed in the longitudinal axis of the culture chamber, said ring (31) having a substantially square or rectangular cross section and comprising bottom (311) and top (312) sides, and inner and outer (313) sides,
i. the bottom, inner and outer sides are formed by leak-tight wails, apart from one or more orifices (34) allowing for the flow of the culture medium in the first flow zone in a horizontal tangential direction,
ii. the top side (312) is perforated so that the culture medium arriving in the first flow zone moves through the perforations in a substantially vertical upward direction to a second flow zone (32),

b) a second flow zone (32), in the form of a ring with substantially square or rectangular cross section, superposed on the first flow zone (31) and comprising leak-tight top (323) and outer (324) sides, an inner side formed by the walls (321, 322) of two concentric cylinders, a first cylinder (36) forming an inner edge (321) which does not meet the top side (323), and a second cylinder (38) with a diameter smaller than the first cylinder and forming an inner edge (322) which does not meet the bottom side, so that the culture medium moves into the space delimited by the walls of the two concentric cylinders in a downward vertical direction, to arrive in a third flow zone (33),

c) a third flow zone (33) delimited by the wails (322) of the second cylinder (38), a leak tight bottom side (331) and a top side formed by the inlet grating (22) in the culture chamber (2), promoting an annular flow of the culture medium in the culture chamber.

The diameters of the first and second cylinders are very close so that the second cylinder can fit into the first cylinder while leaving a space, for example of the order of a few millimeters for a diameter of the cylinders of the same order of magnitude as the bottom inlet diameter of the culture chamber (2).

In a specific embodiment:
the outer diameter of the first flow zone (31) is greater than e inlet diameter of the vertical duct, for example 1.5 to 2 times greater;
the inner diameter (delimited by the first cylinder (36)) is substantially greater than the inlet diameter of the vertical duct forming the culture chamber (2); and
the inner diameter of the second cylinder (38) is equal to the inlet diameter of the vertical duct forming the culture chamber (2).

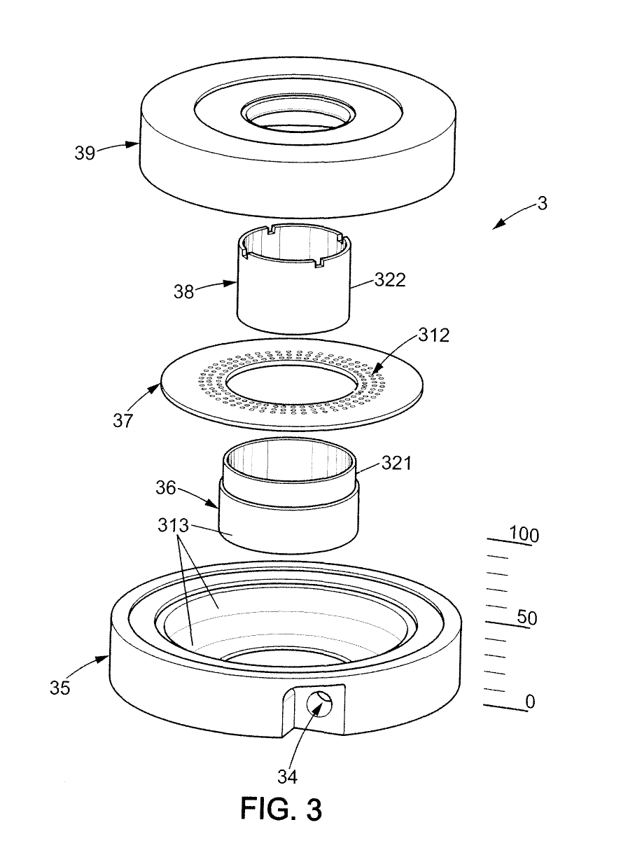

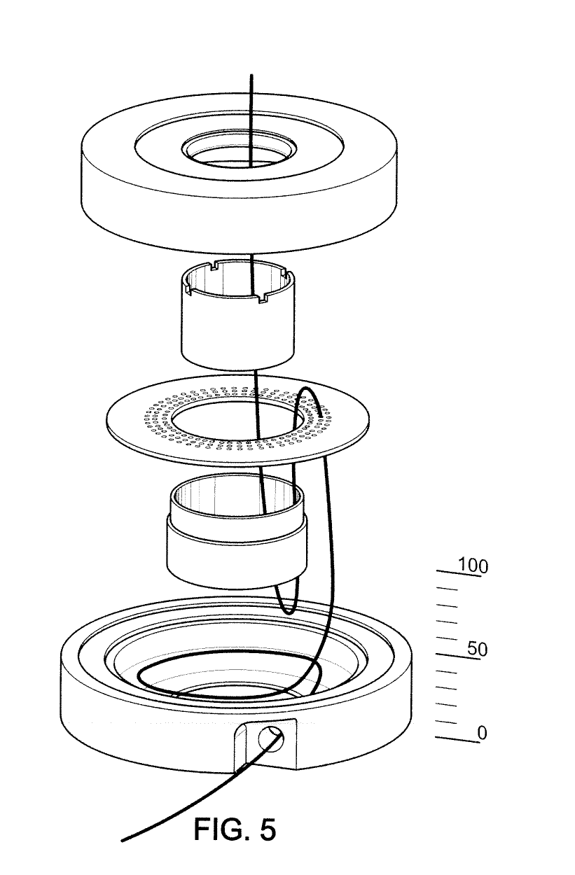

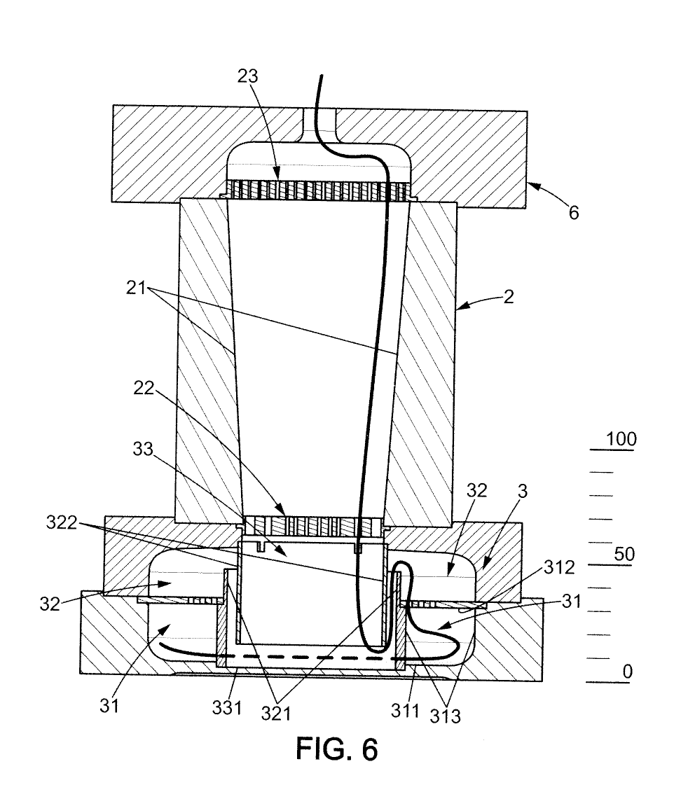

An example of such a device is shown in FIGS. 3, 4 and 6. In this particular embodiment, the path of the culture medium in the device is represented in FIGS. 5 and 6.

The device (3) is described above in the context of an upward vertical flow of the culture medium in the culture chamber. Obviously, a similar device can be used in an embodiment with downward flow.

It will be noted that the essential element for converting, over a short distance, a horizontal centrifugal/tangential flow into a uniform vertical flow, lies in the coupled use of a perforated ring (allowing the fluids to pass only on internal radii where the fluid is rotating at lower velocity) and of a chicane (formed by two concentric cylinders). These elements produce velocity variations and changes of direction which allow for the appropriate reorientation of the fluid.

Also, in another embodiment, there is no separation between the first and second flow zones.

In another embodiment, the bottom wall 311 of the first flow zone has a helical form so that said flow zone changes from a maximum section in line with the fluid inlet orifice 34 (corresponding to the distance between the bottom wall 311 and the top wall 312) with a zero section into a revolution about the vertical axis of the device. Other variants can of course be envisaged that lead to a reduction of the volume of the first flow zone in the main direction of flow of the culture medium.

The Downstream Flow-Creating Device (6)

This device is placed downstream of the outlet grating. It helps to maintain the symmetry of the flow. In a specific embodiment, this device comprises an axial outlet of small diameter (for example connected to a buffer tank) and not posing any problem of prerotation of the flow (which would be the case in the eventuality of a tangential outlet).

The Culture Medium Tank (5)

The tank makes it possible to ensure the conditions of gas exchanges and of renewal of the nutrient liquid (culture medium). The tank must satisfy the usual constraints for sterile cell culture.

If appropriate, it may comprise means for measuring physical quantities such as the pressure, the temperature or the flow rate. It may also comprise catheters or other devices for bringing products into the device (culture medium, etc.) in a sterile manner.

It is also possible to put place a plurality of identical culture medium tanks (which then facilitates the renewal of this culture medium after a certain time of use) or different culture medium tanks (which makes it possible for example to alternate the culture medium by favoring one or other of the phenotypes during the culture). One of the tanks may also be removed from the circuit in order, for example, to transfer the medium into another device, for example a device which would extract the active principles therefrom if the cultivated cells are cells producing biomolecules of interest (proteins, therapeutic antibodies, antivirals, etc.).

Uses of the Bioreactor According to the Invention and Implementation Method

The bioreactors according to the invention are particularly advantageous for cell culture on a three-dimensional support, notably on porous hydrogels. They can in particular be used for:

a) the production of tissue graft, such as bone grafts, notably vascularized bone grafts, cartilage grafts or any other type of tissue/cell (epithelial cells, hepatocytes, granulocytes, erythrocytes, etc., without being limited) or,

b) the production of biological modules, in particular of biopharmaceutical molecules, for example for the production of antibodies or of proteins.

The production of tissue graft in a bioreactor has been described in the prior art for example in the work by Lanza, Langer and Vacanti “Principle of Tissue Engineering”, editions Elsevier.

The bioreactor according to the invention can be used in a method for producing a tissue graft, notably a bone or cartilage graft, comprising the following steps:

a) seeding the porous biomaterial(s) with cells capable of generating a tissue, for example a bone or cartilage tissue, in order to obtain one or more organoids,

b) placing the organoid(s) in the bioreactor according to the invention containing an appropriate culture medium,

c) cultivating the organoid(s) in the bioreactor in conditions appropriate to the formation of said tissue graft, for example of said hone or cartilage graft.

Preferentially, a choice will be made to cultivate one or more organoids of small dimensions in the culture chamber. For example, a choice will be made to cultivate a plurality of organoids of larger section between 2 and 20 mm, for example between 4 and 15 mm, even between 2 and 10 mm, for example each organoid consisting of a fragment of porous hydrogel with a size of a few millimeters, for example between 2 and 20 mm, for example between 4 and 15 mm, even between 2 and 10 mm, for their larger section.

in a preferred embodiment, a plurality of grafts are cultivated in the bioreactor, preferably at least 5 grafts, for example at least 10 grafts, the number and the size of the grafts in culture being able to be adapted, notably as a function of the dimensions of the bioreactor used.

In this preferred culture mode a pulsed flow mode will preferably be chosen in the culture chamber.

It is then advantageously possible to control the flow velocity of the culture medium so that the organoid(s) in culture in the bioreactor are in sustentation in the culture medium at a median vertical position, in particular which makes it possible to avoid having the organoids come into contact with the top and bottom parts of the duct.

In a particular embodiment, the bioreactor allows for the generation of vascularized tissues, for example of vascularized hone tissues. It is possible to cultivate, for example, in co-culture on a porous biomaterial, endothelial progenitor cells and osteoprogenitor cells capable of regenerating a vascularized bone tissue (Unger et al 2007, Biomaterials No. 28 3965-3976).

For the culture methods according to the invention, a porous hydrogel will preferably be chosen, for example from the polysaccharide-based porous hydrogels as described in European Cells and Materials, Vol. 13, Suppl. 1, 2007 (page 50).

The culture medium will be selected according to the targeted objective. Different appropriate culture media for culture on a three-dimensional substrate, notably to obtain bone tissues, are described for example in Lanza, Langer and Vacanti “Principle of Tissue Engineering”, éditions Elsevier.

In another embodiment, the cells cultivated on a three-dimensional support are cells producing biomolecules of interest, for example proteins, notably therapeutic antibodies.

### EXAMPLES

A—Prototype of a Bioreactor According to the Invention

The inventors have produced the following prototype, as represented in FIGS. 2 to 6. The bioreactor comprises
a culture chamber (represented in FIG. 2),
an upstream flow-creating device,
a downstream flow-creating device,
pumping means and a tank.

FIG. 2 represents a detailed view of the culture chamber comprising the culture chamber of cylindrical type (2), the inner walls (21) of which form an inverted cone. At the inlet of the culture chamber, there is a perforated grating (22) promoting the annular flow of the culture medium in the culture chamber. There is also a perforated grating (23) placed at the outlet, preventing the grafts from circulating in the rest of the device.

FIG. 3 represents a detailed view of the upstream flow-creating device and comprises, in particular, a perforated ring (35) allowing the culture medium to flow in an inner radius (31), two concentric cylinders (36) and (38) and a perforated disc (37) and a cap ring (39), the whole forming the chicane promoting an axial flow limiting the horizontal velocity gradient.

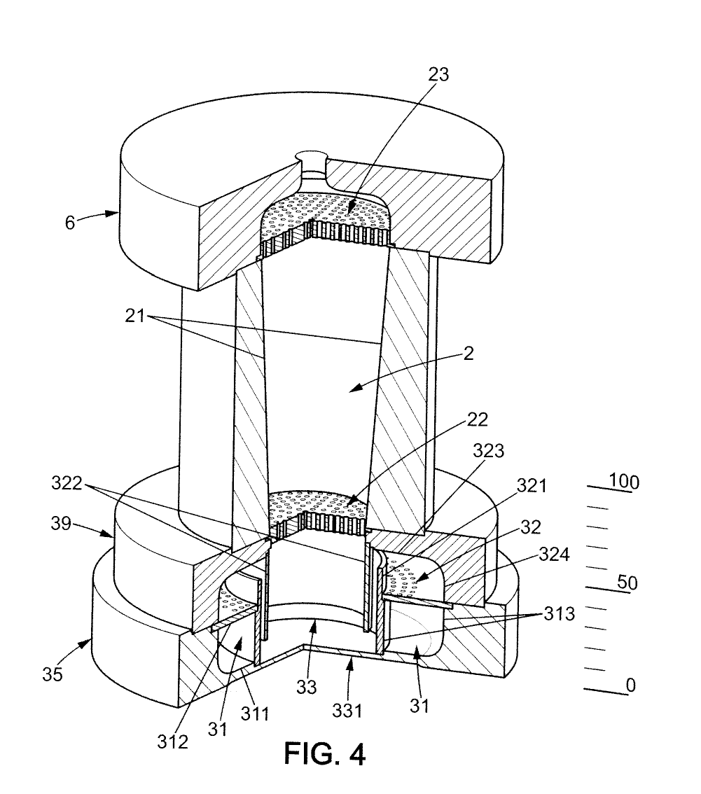

All of these elements are situated in the axis of the culture chamber upstream of the inlet grating (see FIG. 4 for the arrangement of these elements in a partial cross-sectional view).

As represented in FIG. 5, this device makes it possible to convert the flow, over a short distance, from a horizontal centrifugal/tangential inlet to a uniform vertical flow. The fluids move into the inner radius of the ring in a horizontal tangential direction, into a first zone delimited by the inner walls of the perforated ring and the first concentric cylinder. It then passes into a second zone in a vertical direction through the perforations of the perforated disc then drops back between the walls of the first concentric cylinder and the second concentric cylinder to arrive in a third zone to rise up to the inlet grating of the culture chamber.

FIG. 6 shows the arrangement of the constituent elements
of the culture chamber.
of the upstream flow device,
of the downstream flow device,
   and the path of the fluid through these three elements.

B—Bioreactor Tests

The bioreactor, as described in the preceding section, has been subjected to a first series of tests during the course of which mesenchymatous stem cells obtained from bone marrow have been placed in culture on polysaccharide-based porous hydrogels as described in 2007 in European Cells and Materials Vol. 13, Suppl. 1, 2007 (page 50). The nutrient liquid (culture medium) used was IMDM (Iscove's Modified Dulbecco's Medium) with 10% fetal calf serum (commercially available). A mixture of air with 5% CO2 was maintained above the single free surface of the bioreactor circuit (buffer tank).

The tests were conducted in parallel with a similar static culture. The usual culture conditions for cell culture were used.

The results after 24 h and 48 h of culture showed that the cells have a better conformation and better distribution in the hydrogels. An absence of early cell death in culture was also observed in the bioreactor according to the invention, notably at the core of the biomaterials, unlike what is observed with the static bioreactor tests. These results demonstrate a better perfusion of the biomaterials appropriate for issue culture on a three-dimensional substrate.

C—Application of the Bioreactor to Osteoblastic Differentiation of Adipose Tissue Stem Cells (ADSCs)

The bioreactor according to the invention was used to apply dynamic stresses to 3D hydrogel matrixes seeded with adult stem cells originating from adipose tissue (ADSCs) and modulate the osteoblastic differentiation of the ADSCs in 3D, and in the absence of osteoinductive factors.

The cellularized hydrogels (or substrates) were placed in the bioreactor after 48 h of culture in static mode. The dimension of the substrates is approximately 6 mm in diameter and 2 mm in thickness. The hydrodynamic conditions of the pulsed flow generated in the culture chamber of the bioreactor (frequency of 2 Hz, pulsed flow rate varying from 0 to 6.8 L/min with an average flow rate of 3.5 L/min), ensure an adequate sustentation of the 12 cellularized porous substrates placed in this chamber. These flow conditions lead to a dynamic perfusion of the porous substrates with an average flow rate of approximately 6.10⁻⁴ mL/min. After 5 days of dynamic culture, the substrates were harvested for analysis and comparison with the substrates cultivated only in static mode in similar hydrogels and in the same culture medium.

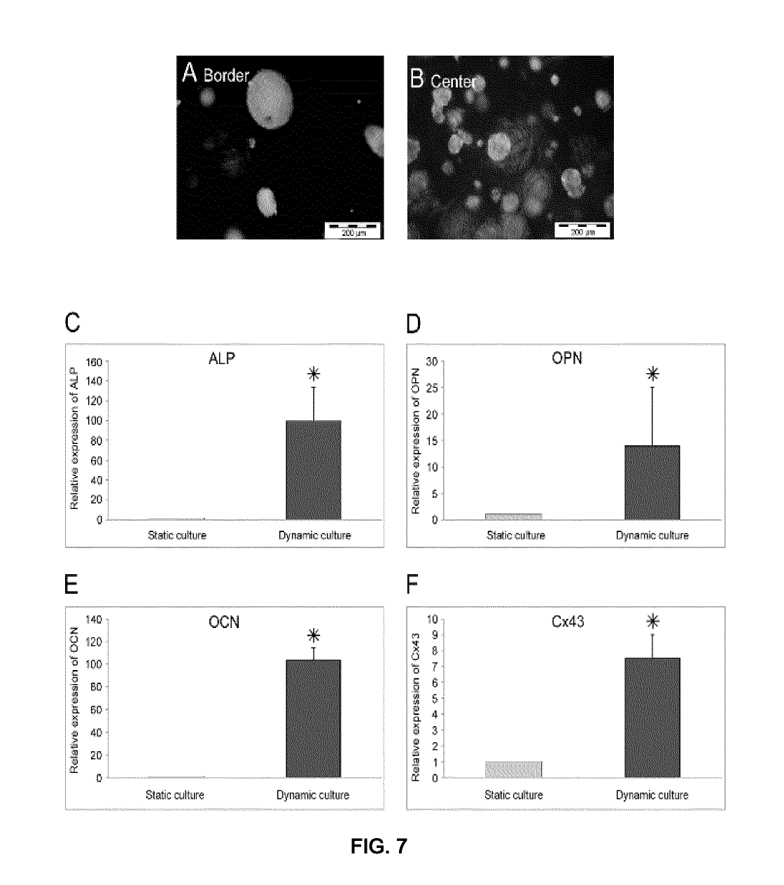

The results of this first study have shown that the differentiation of the ADSCs toward the osteogenic pathway without the addition of osteoinductive factors to the culture medium is increased very significantly after only 5 days of culture in dynamic conditions within the bioreactor, in the absence of any osteogenic factors, with an extremely low perfusion flow velocity (see FIG. 7 and doctoral thesis by Charlotte Lalande, entitled: “Développement d′un nouveau produit d′ingénierie tissulaire osseuse à base de polymères et de cellules souches du tissu adipeux” [Development of a new bone tissue engineering product based on polymers and on adipose tissue stein cells] (23.11.2011)). The cultivated cells are capable of expressing specific bone markers, early (ALP, Col1A1) or late (OCN, OPN), and exhibit a mineralization of the extracellular matrix, even in the absence of osteoinductive factors. The use of the bioreactor could also reinforce the cell-cell interactions, as proven by the increased expression of connexine 43 in these dynamic culture conditions.

1. A bioreactor (1) for cell culture on a three-dimensional substrate, wherein it comprises
a culture chamber (2), the inner walls (21) of which form a vertical duct, with a diameter that widens regularly from the duct inlet to the duct outlet, means (3, 4) enabling the culture medium to flow in said vertical duct.

2. The bioreactor as claimed in claim 1, wherein it also comprises pumping means (4) allowing for a pulsed flow of the culture medium, for example with a pulsing frequency of between 0.05 Hz and 10 Hz.

3. The bioreactor as claimed in claim 1, wherein it comprises means allowing for an annular flow of the culture medium in the culture chamber.

4. The bioreactor as claimed in claim 1, wherein it comprises:
a culture chamber (2), the inner walls (21) of which delimit a vertical duct with a diameter that widens regularly from the bottom inlet of the duct to the top outlet of the duct,
an inlet grating (22) placed in the smaller diameter bottom part of the vertical duct promoting an annular flow of the culture medium,
if appropriate, an outlet grating (23) placed in the top part of the vertical duct,
an upstream flow-creating device (3), upstream of the inlet grating, promoting the annular flow in the vertical duct,
pumping means (4),
if appropriate, a culture medium tank (5) upstream of the culture chamber,
if appropriate, a downstream flow-creating device (6), downstream of the outlet grating.

5. The bioreactor as claimed in claim 1, wherein it comprises an upstream flow-creating device (3), upstream of the culture chamber, consisting of means for converting a horizontal tangential flow into a vertical axial flow of the culture medium.

6. The bioreactor as claimed in claim 1, wherein it comprises an upstream flow-creating device (3), upstream of the culture chamber comprising:
a first flow zone (31), in the form of a ring, the axis of which is placed in the longitudinal axis of the culture chamber, said ring (31) having a substantially square or rectangular cross section and comprising bottom (311) and top (312) sides, and inner and outer (313) sides,
i. the bottom, inner and outer sides are formed by leak-tight walls, apart from one or more orifices (34) allowing for the flow of the culture medium in the first flow zone in a horizontal tangential direction,
ii. the top side (312) is perforated so that the culture medium arriving in the first flow zone moves through the perforations in a substantially vertical upward direction to a second flow zone (32)
a second flow zone (32), in the form of a ring with substantially square or rectangular cross section, superposed on the first flow zone (31) and comprising leak-tight top (323) and outer (324) sides, an inner side formed by the walls (321, 322) of two concentric cylinders, a first cylinder (36) forming an inner edge (321) which does not meet the top side (323), and a second cylinder (38) with a diameter smaller than the first cylinder and forming an inner edge (322) which does not meet the bottom side, so that the culture medium moves into the space delimited by the walls of the two concentric cylinders in a downward vertical direction, to arrive in a third flow zone (33),
a third flow zone (33) delimited by the walls (322) of the second cylinder (38), a leak-tight bottom side (331) and a top side formed by an inlet grating (22) in the culture chamber (2), promoting an annular flow of the culture medium in the culture chamber.

7. The bioreactor as claimed in claim 1, wherein the vertical duct of the culture chamber is tapered and the angle at the apex of the cone of the vertical duct does not exceed 8°.

8. The bioreactor as claimed in claim 1, wherein it comprises an inlet grating (22) placed in the smaller diameter part of the vertical duct promoting an annular flow of the culture medium, the grating having orifices, preferably with a diameter less than 6 mm, for example from 2 to 5 mm, distributed in such a way that the flow is faster in the regions close to the walls than at the center.

9. (canceled)

10. (canceled)

11. A method for producing a tissue graft, comprising the following steps:
seeding one or more porous biomaterials with cells that regenerates a tissue, for example a bone or cartilage tissue, in order to obtain an organoid,
placing the organoid or organoids in a bioreactor as claimed in claim 1 containing an appropriate culture medium,
cultivating the organoid or organoids in the bioreactor in conditions appropriate to the formation of said tissue graft.

12. The method as claimed in claim 11, wherein the culture medium exhibits a pulsed annular flow in the culture chamber.

13. The method as claimed in claim 11, wherein the speed of flow of the culture medium in the culture chamber is controlled so that the organoid or organoids in culture in the bioreactor are in sustentation in the culture medium without coming into contact with the inlet or the outlet of the culture chamber.

14. The method as claimed in claim 11, wherein endothelial progenitor cells and osteoprogenitor cells that regenerates a vascularized bone tissue are cocultivated on a porous biomaterial.

15. The method as claimed in claim 11, wherein the biomaterial is a porous hydrogel.

16. The method of claim 11 for producing a bone or cartilage graft.

17. The method of claim 15, wherein said porous biomaterial is a polysaccharide-based porous hydrogel.

18. The bioreactor of claim 1, wherein said vertical duct is tapered.

19. A method for culturing cells on a three dimensional support, comprising culturing said cells on a three dimensional support in a bioreactor as claimed in claim 1.

20. The method of claim 19, wherein said three dimensional support is a porous hydrogel.

21. The method of claim 19, for producing biological molecules.

22. The method of claim 21, wherein said biological molecules are biopharmaceutical molecules.

23. The method of claim 22, wherein said biological molecules are antibodies or proteins.
